(19) United States
(12) Patent Application Publication
(10) Pub. No.: US 2011/0060312 A1
(43) Pub. Date: Mar. 10, 2011

(54) CONNECTION SYSTEM FOR CONNECTING A FLUID LINE TO A FLUID RESERVOIR

(75) Inventor: Simon Scheurer, Bern (CH)

(73) Assignee: ROCHE DIAGNOSTICS INTERNATIONAL AG, Steinhausen (CH)

(21) Appl. No.: 12/869,217

(22) Filed: Aug. 26, 2010

(30) Foreign Application Priority Data
Feb. 29, 2008 (EP) 08003777.3
Feb. 25, 2009 (EP) PCT/EP2009/001322

(51) Int. Cl. A61M 25/00

(52) U.S. Cl. 604/523

(57) ABSTRACT

A connection system for connecting a fluid line to a fluid reservoir located within a device by way of a cannula and a method thereof are disclosed. The connecting system may include a cannula holder, a movable cannula cover, and lock for the cover. The system may further include a receiving element for receiving the cannula holder; the receiving element may have a release adapted for interaction with the lock. The release and the lock can be arranged to release the cannula cover by the release interacting with the lock, so that the cannula cover is movable by the inserting force to expose the cannula when the cannula holder is inserted into the receiving element. Thus, a connection system is provided for easy and secure connection without danger of injury by the cannula or staining of the cannula.

### TECHNICAL FIELD

The disclosure relates to a connection system for connecting a fluid line to a fluid reservoir located within a device, and in particular within a medical device such as, e.g., an insulin pump.

### BACKGROUND

It is known to connect a fluid line to a fluid reservoir by way of adapted connectors. The reservoir may be an ampoule provided with a connector to which a so-called Luer connector fixed to one end of the fluid line can be attached. Such connection systems are, for example, used in medical pumping devices, e.g., portable insulin pumps, wherein the ampoule contains insulin is placed within the pump. The insulin is dosed by the pump and fed via the fluid line to the patient in a known manner. An improved connection system is still desired.

### SUMMARY

In one embodiment, a connection system for connecting a fluid line to a fluid reservoir located within a device by way of a cannula is disclosed. The connection system may include a cannula holder holding the cannula and comprising a movable cannula cover and a lock for the cover, which prevents the movement of the cannula cover in a first state covering the cannula. A receiving element which receives the cannula holder can be provided, wherein the receiving element can be fixed to the device or formed a part thereof. The receiving element may comprise a release which interacts with the lock, and wherein the release and the lock are arranged to release the cannula cover by the release interacting with the lock, so that the cannula cover is movable by the inserting force to expose the cannula when the cannula holder is inserted into the receiving element, and so that the cannula cover is in a second state exposing the cannula when the cannula holder is inserted within the receiving element.

In another embodiment, a medical pumping device is disclosed having a receiving element of a connection system according to any one of the embodiments described herein.

In still another embodiment, a method of connecting a fluid line to a fluid reservoir located within a device by way of a cannula is disclosed. The method comprises utilizing a connecting system according to any one of the embodiments described herein.

These and other features of the various embodiments of the invention will become apparent from the following detailed description and drawings.

### BRIEF DESCRIPTION OF THE DRAWINGS

In the figures, like reference numerals depict the same or functionally equivalent elements, wherein:

FIG. 1 shows a first embodiment of a connecting system wherein a receiving element is an adapter to be fixed to a device;

FIG. 2 shows a sectional view of the cannula holder of FIG. 1 with a cannula cover in a first state covering a cannula;

FIG. 3 shows the cannula holder of FIG. 2 with the cannula cover in the second state exposing the cannula;

FIG. 4 shows a sectional view of the cannula holder of FIGS. 1 to 3 ready to be inserted in the receiving element of the system;

FIG. 5 shows a sectional view of the cannula holder of FIG. 4 inserted into and fixed to the receiving element and with a pierced ampoule;

FIG. 6 shows a sectional view similar to FIG. 4, but rotated 90 degrees about the longitudinal axis of the cannula so that a lock and a release can be seen;

FIG. 7 shows the sectional view of FIG. 6 with the cannula holder inserted within the receiving element;

FIG. 8 shows a perspective view of a cannula cover with parts of a lock;

FIG. 9 shows a perspective view of a part of a cannula holder with other parts of a lock;

FIG. 10 shows a sectional view of a cannula holder with a cannula cover and a part of a receiving element with a release;

FIG. 11 shows a sectional view of a part of a cannula holder inserted into a receiving element;

FIG. 12 shows a sectional view of a device with a connection system according to a second embodiment with a cannula holder and a receiving element, wherein the receiving element forms part of the device itself;

FIG. 13 shows the cannula holder and the device of FIG. 12 in separated state before loading of the ampoule into the device;

FIG. 14 shows a first step of the opening of the device of FIG. 13;

FIG. 15 shows a second step of the opening of the device of FIG. 14;

FIG. 16 shows the insertion of an ampoule into the device of FIG. 15;

FIG. 17 and FIG. 18 show the closing of the device of FIG. 16;

FIG. 19 shows a cannula holder in perspective view in exploded and assembled form;

FIG. 20 shows an exploded view for explaining a blocking element of the connection system according to the second embodiment;

FIG. 21 shows the elements of FIG. 20 in assembled form;

FIG. 22 shows an unblocking of a cannula holder;

FIG. 23 shows blocking and unblocking elements of a third embodiment in exploded view;

FIG. 24 shows the blocking and unblocking elements of FIG. 23 in a sectional view;

FIG. 25 shows in exploded view a connection system according to another embodiment with a form which locks rotational movement of a cannula cover; and

FIG. 26 shows the embodiment of FIG. 25 in sectional view before inserting the cannula holder into a receiving element.

### DETAILED DESCRIPTION

In one embodiment, a connection system for connecting a fluid line to a fluid reservoir located within a device by way of a cannula is disclosed. The system may comprise a cannula holder holding the cannula, a movable cannula cover, and a lock for the cover which prevents movement of the cannula cover in a first state which covers the cannula. The system may also comprise a receiving element for receiving the cannula holder. The receiving element may be either adapted for fixation to the device or form part of the device. The receiving element may comprise a release adapted for interaction with the lock. The release and the lock may be arranged to release the cannula cover by the release interacting with the lock, so that the cannula cover is movable by the inserting force to expose the cannula when the cannula holder is inserted into the receiving element, and so that the cannula cover is in a second state exposing the cannula when the cannula holder is inserted within the receiving element. By this connection system the cannula is protected automatically and gives, on the one hand, secure operation to the operator thereby preventing injury risks by the cannula, and prevents, on the other hand, staining of the cannula. The connection system can be easily adapted for one-hand operation.

In another embodiment of the connection system, while the cannula holder is being removed from the receiving element, the cannula cover is moved by an energy storage device in a direction opposite to the movement of the cannula cover during insertion of the cannula holder into the receiving element. This helps considerably for a one-hand operation if the energy storage element is selected such that it actively pushes the cannula holder out of the receiving element when the connection is released again. If the connection accidentally becomes loose, it will disconnect by itself if the energy storage element is selected for providing enough force, so that a connection that has become loose-fitting by accident is released. In one embodiment, the cannula cover is held slidably within the cannula holder for a sliding translation movement in longitudinal direction of the cannula. This embodiment allows for a very compact connection as the cannula cover does not have to have a size which takes up the collar of the reservoir. In one embodiment, the energy storage device may be a coil spring.

In an alternative embodiment to the energy-storing element, the cannula cover is held within the cannula holder by a guide imparting a rotational screw-in and screw-out movement to the cannula cover within the cannula holder during the insertion and the removing of the cannula holder from the receiving element. In one embodiment the lock comprises a catch on the cannula cover which is adapted to lock into a recess of the cannula holder, and the release comprises a rib within the receiving element which is adapted to release the catch from the recess. The catch may be a two-stepped catch comprising a first block-shaped step and a second step which is held by an elastically deformable tongue. In one embodiment, the connection system may comprise blocking elements for blocking of the cannula holder after full insertion into the device and unblocking elements for unblocking of the cannula holder and for releasing the cannula holder from the device. In another embodiment, an unblocking element is provided for one-handed operation of the release, which allows for easy operation of the connection system. The receiving element in one embodiment is part of a medical device, which obviates the need for a separate adapter. In such an embodiment, the medical device takes up the forces in connection with the pumping of the liquid, which allows a simpler construction of the connection. In one embodiment, the receiving element may be a part of an insulin pump. In still another embodiment, the cannula holder is essentially L-shaped, wherein the cannula is held essentially in a 90 degree angle to the line exiting the cannula holder. This embodiment allows a full integration of the connection system into the shape of the medical device and results in an advantageous exit of the fluid line. In another embodiment, the receiving element may be mounted to the device such that it can be swiveled about an axis for opening a chamber that is adapted to receive the liquid reservoir. This embodiment, as well, allows for a good adaptation of the connection system to a portable fluid pump.

Other embodiments of the invention further relate to a medical pumping device with a receiving element according to the invention. This medical pumping device may be a portable medication pump in one embodiment and in particular, an insulin pump in another embodiment.

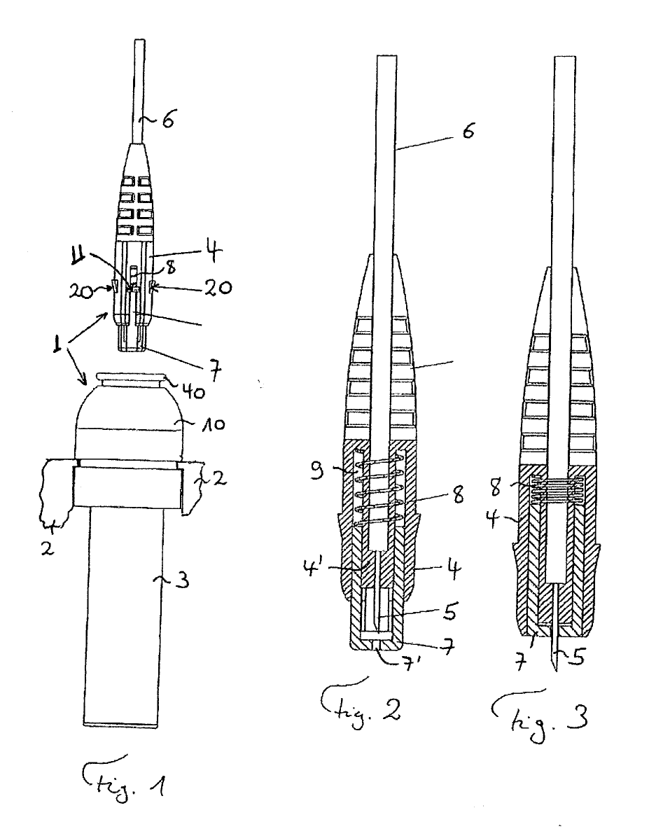

With reference to FIGS. 1 to 11, a first illustrated embodiment of the invention is now described. FIG. 1 shows an overview of this first illustrated embodiment of the connecting system 1. There is provided a cannula holder 4 holding a cannula that cannot be seen in FIG. 1 since this cannula is covered by cannula cover 7 which prevents an accidental touching of the cannula by the user of the system. Within cannula holder 4, the cannula is in fluid connection with line 6 which shall be connected by the system to a reservoir 3 containing the fluid. This reservoir, in particular an ampoule, for example, containing insulin, is provided within a device 2 which is only indicated in this embodiment by a part of its housing.

This device is, for example, a medical device in one embodiment and in particular, an insulin pump in another embodiment for dosing insulin out of reservoir or ampoule 3, respectively, to a patient via tube or line 6 that leads to another cannula at its other end (not shown) for insertion into the body of the patient. The system according to this embodiment comprises a receiving element 10 into which the cannula holder 4 is to be inserted in the form of an adapter, which is shaped so that it fits the device 2, and preferably surrounds the upper part of the reservoir 3 in another embodiment. The cannula 5 (FIG. 2) within the cannula holder 4 is inserted into the reservoir 3 when the cannula holder 4 is inserted into the adapter or receiving element 10 in this embodiment. A lock, generally shown as 11, prevents the cannula cover 7 from being moved or released, respectively, unless upon insertion of the cannula holder 4 into the receiving element 10.

Upon insertion, a release within the receiving element 10 interacts with the lock 11 so that the cannula cover 7 moves. In one particular embodiment, the cannula cover 7 slides backwards, away from the tip of the cannula 5, to expose the cannula within the receiving element 10, so that the reservoir 3 can be pierced by the cannula 5 to establish the fluid connection between the reservoir 3 and line 6. In case of an ampoule, a septum closing the ampoule is pierced by the cannula. One or more blocking elements, generally depicted as 20 in FIG. 1, lock the fully inserted cannula holder 4 within the adapter or receiving element 10. An unblocking element, part of which is generally shown as 40 in FIG. 1, serves for releasing the cannula holder 4 from receiving element 10. Upon releasing the cannula holder, the cannula cover 7 is again brought in position as shown in FIG. 1 to cover the cannula so that no contact of the person using the system with the cannula is possible, as will be explained in greater detail below. By this arrangement, the person is protected against injury by the cannula on the one hand and on the other hand, the cannula is protected against contamination.

The cannula holder 4 of this embodiment is generally shown as having a cylindrical outer shape and consequently the cannula cover 7 and the inner shape of the receiving element 10 receiving the holder is shown cylindrically as well. Of course, other shapes can be selected as well. The cannula holder 4 and the receiving element 10 of the system 1 will usually be made of plastic material. In some embodiments, the cannula holder 4 and the receiving element 10 are preferably one-piece elements as shown in the drawings, but may of course as well be multi-piece elements in other embodiments.

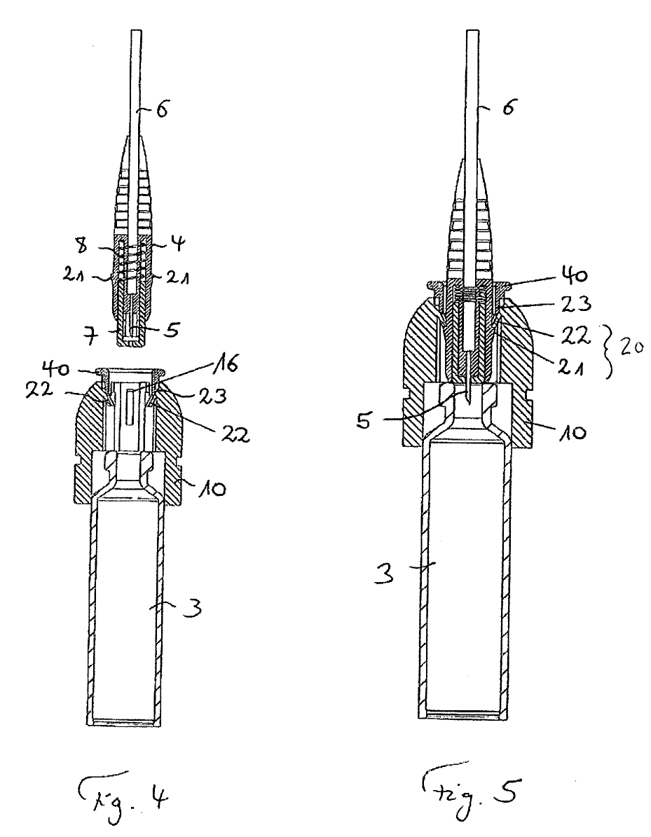

FIGS. 2 and 3 show a sectional view of the cannula holder 4 of the first embodiment of the connection system 1. In these sectional views, the cannula 5 can be seen which is fixedly mounted within the cannula holder 4, for example by being fully enclosed by plastic core 4′ of this cannula holder. Within holder 4, the cannula 5 is in fluid connection with line 6, so that fluid entering the cannula at its tip enters the line 6. The cannula cover 7 fully covers cannula 5 in the first position or state, respectively, shown in FIG. 2. The cover 7 is locked in this position by the lock 11, which is not visible in this section, until the release of the receiving element 10 (FIG. 1) releases the lock, as will be explained below. When the lock 11 (FIG. 1) is released, the cover 7 can be moved.

In one particular embodiment, the cannula cover 7 can slide backwards within space 9 in the cannula holder 4, so that the cannula 5 becomes exposed as can be seen in FIG. 3, which shows the cover pushed fully backwards; because of the lock 11 (FIG. 1), in this second position or state, respectively, shown here in FIG. 3 for explanation purposes, the “free” cannula holder 4 can only be assumed when this holder is inserted within the receiving element 10 of the connection system 1. The cannula 5 passes through the small opening 7′ of the cannula cover 7 during the backwards movement of the cannula cover. Preferably, an energy storage element 8 is provided which stores energy that becomes available by the insertion force applied by the user of the system 1 upon inserting the cannula holder 4 into the receiving element 10. In some embodiments, the energy storage device 8 is a coil spring.

When the cannula holder 4 is again removed from the receiving element 10, the stored energy is available to push the cannula cover 7 back into the first position covering the cannula 5 as shown in FIGS. 1 and 2, such that the lock 11 will again lock the cover 7 in this position. In some embodiments, the force exerted by the coil spring or energy storage device 8 on the cannula cover 7 may be selected to be relatively low by a corresponding spring size and spring rate. In this manner, this force of the energy storage device 8 serves essentially only to push the cannula cover 7 back over the cannula 5 when the cannula holder 4 has been unblocked by the user and is pulled out of the receiving element 10. On the other hand, and preferably in one embodiment, the spring force is selected as being so great that the spring or energy storage device 8 acting on the cannula cover 7 significantly helps pushing the cannula holder 4 out of the receiving element 10 by the cannula 7 cover abutting on the receiving element 10 when the unblocking element 40 has been actuated by the user.

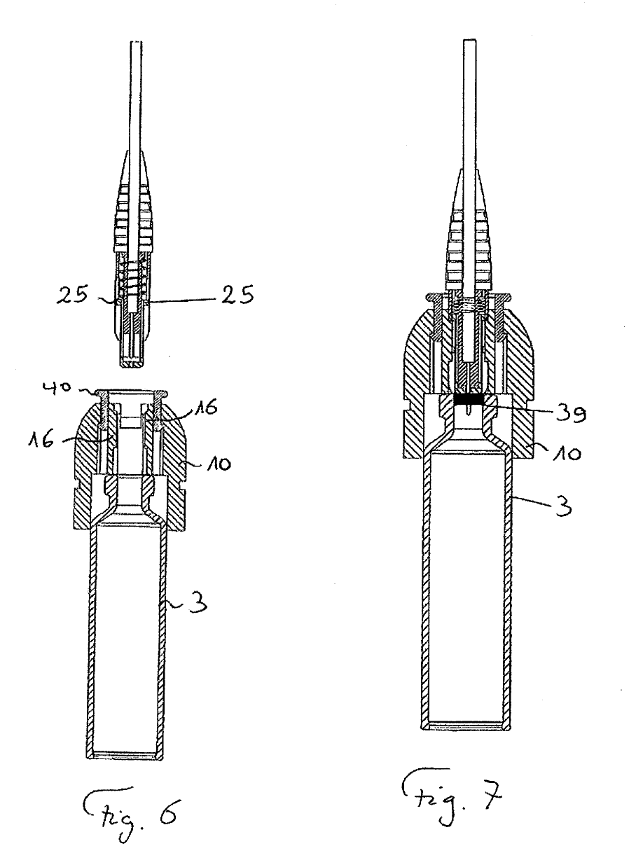

FIGS. 4 and 5 show the insertion of cannula holder 4 into the adapter or receiving element 10, which is depicted in this embodiment within device 2 and in sectional view. In the sectional view of FIG. 4, a part of the release within receiving element 10 can be seen in the form of an elongated protruding member or rib 16 that interacts with the lock 11 for the cannula cover 7 of cannula holder 4 when the holder 4 is inserted into the receiving element 10. In the section of FIGS. 4 and 5 the lock 11 (FIG. 1) cannot be seen, but will be shown and explained below with reference to FIGS. 6 to 11. On the one hand, FIGS. 4 and 5 show the insertion of the cannula holder 4 and the piercing of the reservoir 3, which in this embodiment is provided as an ampoule, by the cannula 5 in the inserted position (FIG. 5). On the other hand these Figures show the blocking elements 20 for securing the inserted holder 4 within the receiving element 10 and the unblocking element 40 for the release of the holder 4 from the receiving element 10 by the user, which is usually done when an empty ampoule or reservoir 3 and/or the line 6 with the cannulae on both ends have to be replaced. In this embodiment, the blocking elements 20 are provided by interacting protrusions on the outside of the cannula holder 4 and on the inside of the receiving element 10. In particular, wedge shaped protrusions 21 are provided on the outer circumference of the holder 4 and corresponding latches 22 are provided on the inner circumference of the receiving element 10. The interaction of the protrusions 21 and latches 22 allows to insert the cannula holder 4 into the receiving element 10 and to block the holder in the fully inserted state. In the shown example, the latches 22 are bended by the wedge shaped protrusions 21 during the insertion and spring back (into their normal unbent positions shown in FIGS. 4 and 5) in the fully inserted state of the cannula holder 4. In the fully inserted state, the latches 22 contact the horizontal planes of the wedge shaped protrusions 21, thus blocking the holder 4 in the fully inserted state. Several such interacting protrusions 21 and latches 22 may be arranged around the holder 4 and the receiving element 10. The holder 4 and the receiving element 10 may be keyed, for example by a corresponding guide, so that the insertion can only be done in a position where the interacting protrusions align. This guiding is preferably provided by the interacting lock and release for the cannula cover 7, but other matching keying elements can be provided to allow an insertion in a correct position only, so that interaction of the blocking elements occurs.

FIGS. 4 and 5 show as well the unblocking element 40, which is in this example is provided as a collar that is slidably secured to the receiving element 10 and is provided with a ring or fingers 23 at the positions of the latches 22, that bend the latches 22 in radial direction away from the wedge shaped protrusions or wedges 21 when the collar or unblocking element 40 is moved by finger pressure by the user in direction towards the cannula tip. In this manner, the latches 22 no longer block the wedges 21, and thus the user can pull the cannula holder 4 out of the receiving element 10. The pulling out is helped more or less, as outlined above, by the force of coil spring or energy storage device 8 that at the same time presses cannula cover 7, abutting on ampoule 3, over the cannula 5 during the pull-out movement of the cannula holder 4.

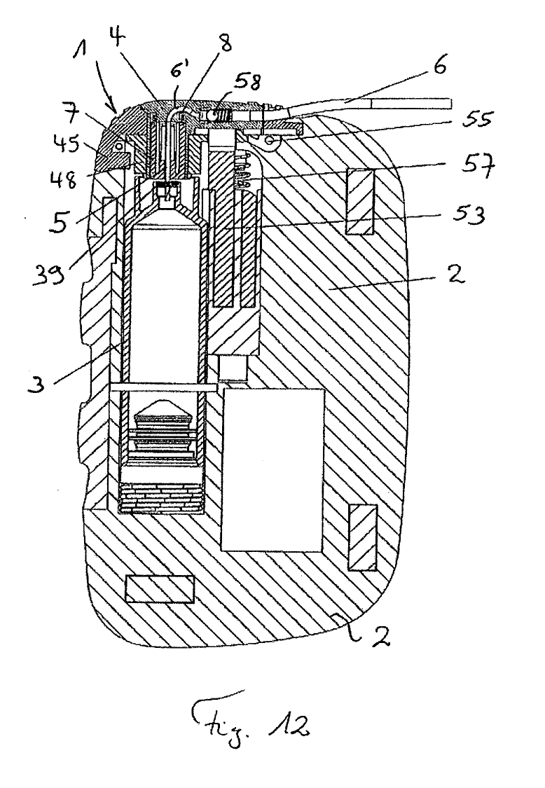

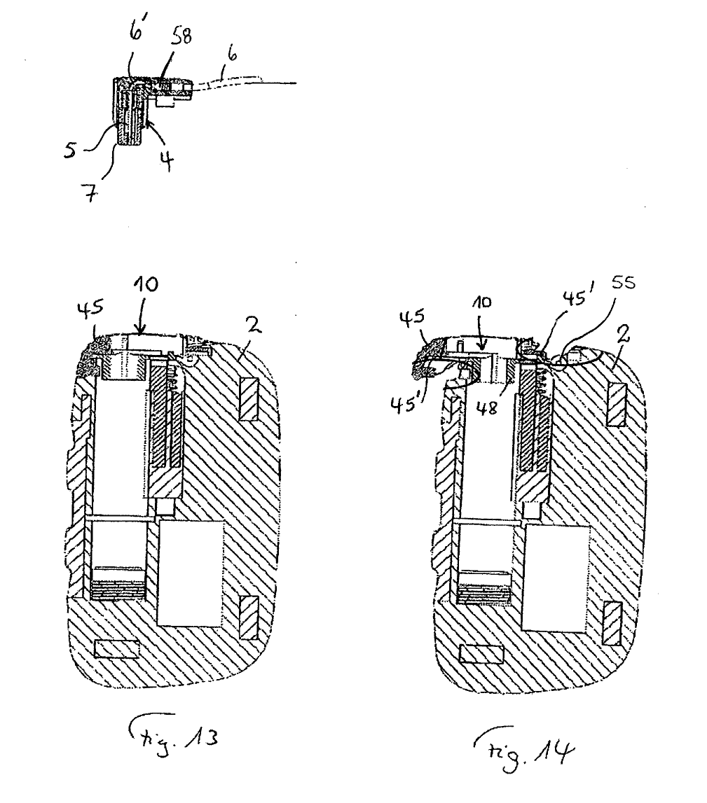

With reference to FIGS. 6 to 11, the lock and the release for the cannula cover 7 can now be described in greater detail. FIGS. 6, 7, 10 and 11 show sectional views wherein these means can be seen and FIGS. 8 and 9 show perspective views of the cannula cover 7 and the main part of the cannula holder 4 for a better explanation. Further reference is made to FIG. 4 wherein a part of the release is shown by rib 16 already mentioned. In one embodiment, the cannula cover 7 is provided with a two-step catch 25 having a narrower, wedge shaped step 26 and a wider, block-shaped step 27 and in other embodiments, with two such catches 25 that may be arranged opposed each other along the circumference of the cover 7. In still other embodiments, two or more such catches 25 may be provided around the circumference of the holder 4. In some of the embodiments, each catch 25 is formed at the end of an elastic tongue 28, which allows the catch to be bent in inward direction of the cannula cover 7, as can be seen in particular in FIG. 11, and to spring back into its rest position shown in FIGS. 6, 8 and 10. This tongue 28 and the catch 25 is preferably in one embodiment, in one piece with the wall 29 of the cannula cover 7 by providing slits into the wall which define the tongue. Further protruding ribs 30 may be provided on the outside of this wall 29 to provide sliding guides for the cannula cover 7 within the holder 4 that is provided in this case with corresponding groves or slits taking up these sliding guides.

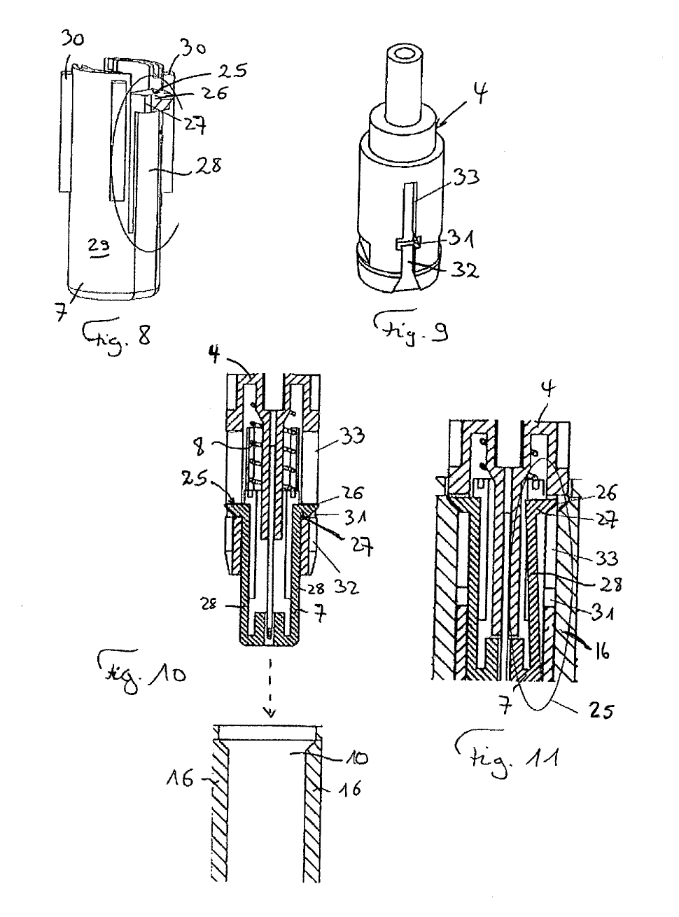

The cannula holder 4 is provided with a hole or recess 31 for taking up the block-shaped step 27 of catch 25 (and in the case of several catches/tongues is provided with the same number of corresponding holes 31 for the block-shaped steps of these catches). In one embodiment, the recess 31 may be a hole. When the cannula cover 7 is mounted in the cannula holder 4 and the cover 7 is in the first locked state covering the cannula, then the block-shaped step 27 of catch 25 sits in the correspondingly shaped hole 31. In this position, for example, shown in FIGS. 1 and 6, the cannula cover is thus locked against movement. When the cannula holder 4 is inserted into the adapter or receiving element 10, then the release will remove the catch 25 from this locked position, allowing the moving of the cannula cover 7 during the insertion of the cannula holder 4 into the receiving element 10. In the shown example, the rib 16 (and in the case of several catches 25, the correspondingly placed several ribs 16) on the inside of the receiving element 10 first slides within a groove 32 of the cannula holder 4 leading to the hole 31. Then, when the insertion of the cannula cover 7 into the receiving element 10 proceeds, rib 16 contacts the wedge-shaped, narrower step 26 of catch 25 that protrudes into groove 32 (FIG. 10). Rib 16 then forces the catch 25 inwardly (e.g., in direction towards the cannula 5), thereby elastically deforming the tongue 28, which frees the block-shaped step 27 from hole 31 such that the cover 7 can slide upwards (away from the tip of the cannula 5). During the upward movement step 26 of catch 25 is sliding within longitudinal slit 33 of the cannula holder 4 that connects to hole 31. When the cannula cover 7 is fully moved backwards (which, as well, corresponds with the full insertion and the blocking of the cannula holder 4 in the receiving element 10 by the blocking element 20 as explained above) the position of FIG. 11 is taken in which the rib 16, the wedge-shaped, narrower step 26, and the inwardly bent tongue 28 (bending shown only for the right tongue in the Figure) can be seen. When the blocking elements 20 are unblocked and the cannula holder 4 is pulled out of receiving element 10, rib 16 moves accordingly downwards (in direction towards the tip of the cannula) and the cannula cover 7 is pressed downwardly by the coil spring or energy storage device 8 as well. During this downward movement, catch 25 again reaches hole 31 where the block-shaped step 27 of catch 25 again enters the hole 31 due to the spring back elasticity of the tongue 28. Thus, cover 7 moves during the pull out movement over the cannula 5 again, covering the same more and more until once again the cover 7 is locked in the state of fully covering the cannula 5 when the cannula holder 4 is fully removed from the receiving element 10. Thus, a fool prove cannula cover results having a fully automatic function both during insertion of the cannula holder into the receiving element and during pulling out of the cannula holder from the adapter or receiving element 10, and thus also into and from device 2. In the fully inserted state shown in FIGS. 5 and 7, the cannula 5 has pierced the reservoir 3 (and in case of an ampoule, the septum 39 thereof) so that a fluid connection between reservoir 3 and line 6 is provided by means of the cannula 5.

With reference to FIGS. 12 to 21, another embodiment of the invention is now described. Same reference numerals as before depict same elements or functionally equivalent elements. In this illustrated embodiment, the connection system 1 may comprise a receiving element 10 that is not an adapter but rather forms a part of the device 2 itself. FIG. 12 shows a sectional view through a device 2, but of which only the functional elements relevant for the connection system are shown. In one embodiment, the device 2 may be a portable medical pump, and in another embodiment, the device 2 may be an insulin pump. Within the device, 2 a reservoir 3 in form of an ampoule can be placed which is in fluid connection with line 6′, 6 by means of cannula 5. It is to be appreciated that the cannula 5 pierces the septum 39 of the ampoule or reservoir 3 when the cannula holder 4 is inserted into the receiving element 10, which may be also part of device 2. The building elements of this embodiment are now explained in greater detail.

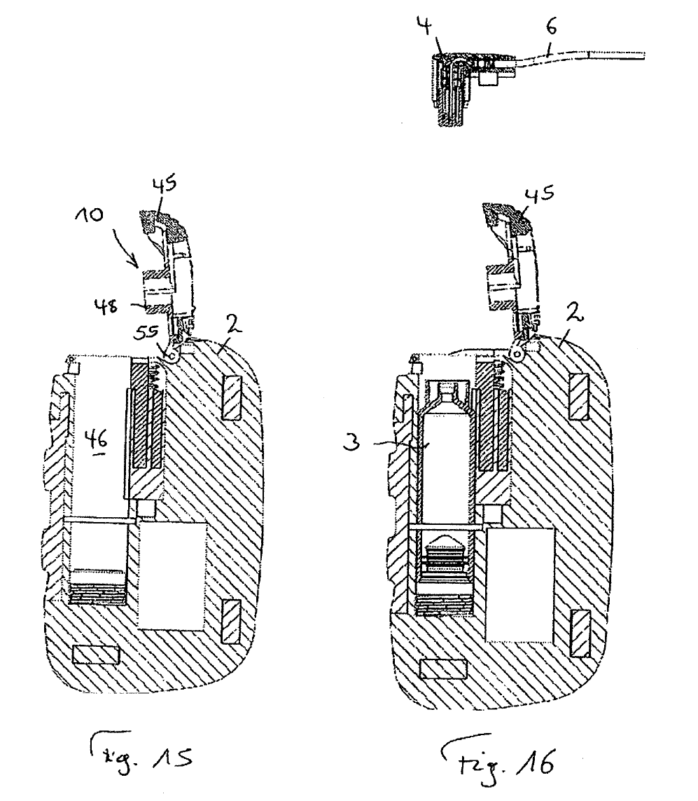

FIG. 13 shows the “empty” device 2 without an ampoule 3 and the cannula holder 4 pulled out of the device. The cannula holder 4 has essentially an L-shape wherein the cannula 5 stands in a 90-degree angle to the line 6 as its exits the holder 4. The straight cannula 5 is connected to the line 6 for example via a curved conduit 6′ and a valve 58 may be present between the conduit 6′ and line 6, allowing a fluid flow only by pressure of the pump. The part of the holder 4 where the cannula 5 is held and the cannula cover 7 are still essentially cylindrical parts but may be of different shape as well.

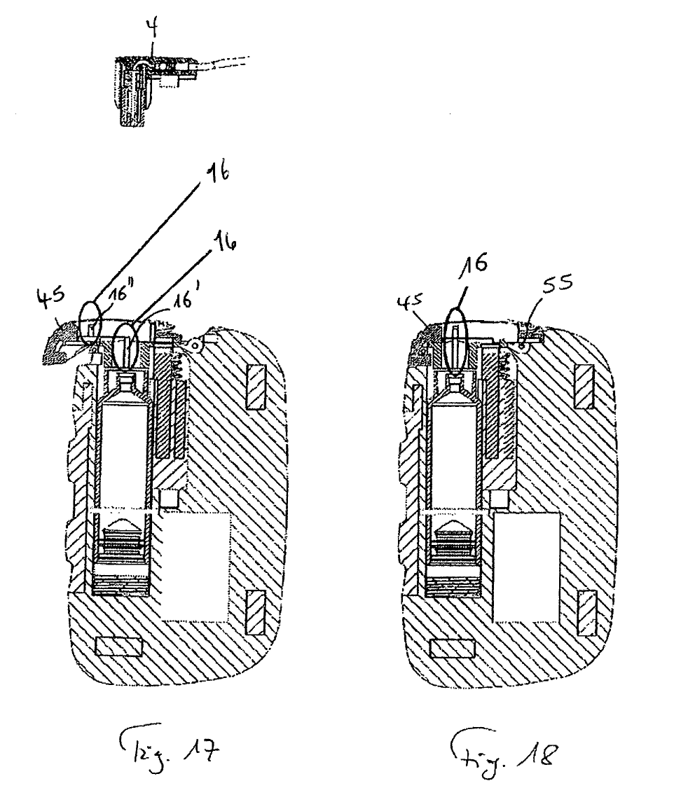

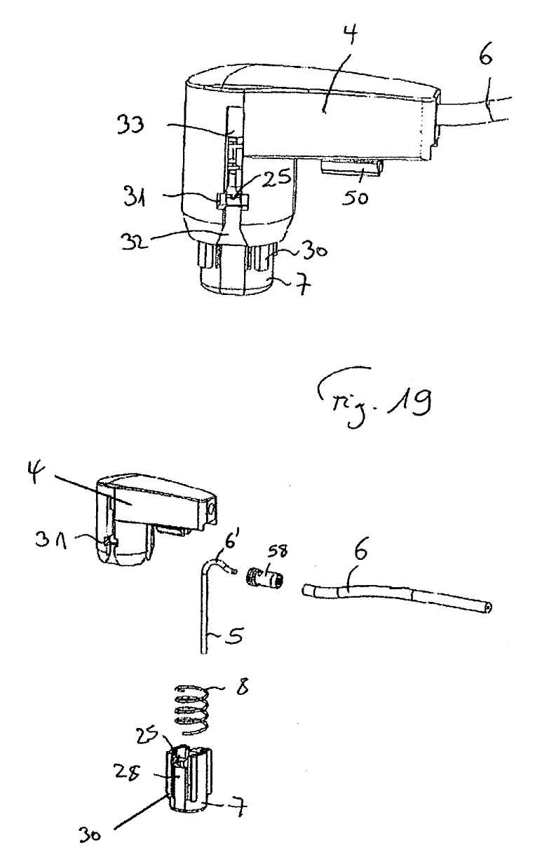

As shown in FIG. 14, the receiving element 10 may comprise, for example, a sliding lid 45 which is slidable with regard to the part 48 for taking up the cannula holder 4. The sliding lid 45 may comprise catch mechanisms 45′ for holding the sliding lid 45 shut and for allowing the opening thereof by the user. As the design of mechanisms 45′ for a lid is well known to person in the art, no further explanation is provided herein. The preferred receiving element 10 of this embodiment is further provided with an axis 55 that, after sliding the lid 45 to its opening position allows a swiveling motion of the receiving element 10 (FIG. 15) to open the reservoir chamber 46 of the device 2 into which the ampoule can be inserted (FIG. 16). The receiving element 10 is then swiveled back (FIG. 17) and the lid 45 is slid closed, so that the ampoule 3 is enclosed in the device (FIG. 18). In this embodiment, the release uses at least one rib 16 as well, but this rib is provided by two rib sections 16′ and 16″ on the ampoule seat 48 and the sliding lid 45, respectively. The two rib sections 16′ and 16″ align to rib 16 when the lid 45 is closed (FIG. 18). The lid 45 is held in its closed state for example by snapping means as catch mechanisms 45′. The pump user can use any kind of closing means that allow an easy closing and of course opening of the reservoir chamber 46. At the same time, the receiving element 10 and the closing means on this element must obviously be designed to hold the ampoule or reservoir 3 firmly in place while the pump is pressing liquid out of the ampoule and—via cannula 5—into line 6. The pump is, for example, working by a motor and spindle drive for a piston provided within the ampoule at the other end of the ampoule facing away from the cannula. This way of driving the fluid out of the ampoule is known in the medical pump art and is therefore not explained in detail here. The design of a cover for the chamber 46 that can be opened and closed, and which at the same time provides the second part 10 of the connection system for inserting the cannula holder 4 and holding the ampoule or reservoir 3 in place, can be chosen in different ways by the person skilled in the art. Accordingly, as explained so far and shown in the drawings, the sliding lid 45 and swivable element 10 is a preferred example only.

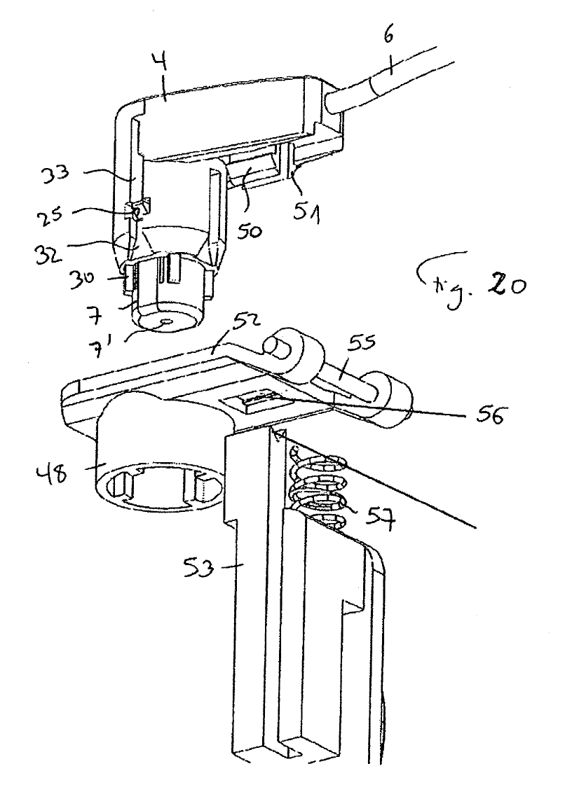

Now, referring again to FIG. 18, the rib 16 is ready to act on the lock for the cannula cover 7 of the cannula holder 4. The rib 16 is an example for the release in one embodiment, which in some embodiments, at least two of such ribs facing each other on the inner circumference of the cylindrical seat 48 for the cannula holder are provided. In the illustrated embodiment of FIG. 18, the lock is preferably constructed identically as explained in the former embodiment of FIGS. 1 to 11. Thus, for the second embodiment shown by FIG. 19 (showing an exploded view and an assembled view of cannula holder 4 in the same Figure), the cannula holder is shown with the two stepped catch 25 within hole 31 as well and with groove 32 for the rib and slit 33 for the second, narrower step 26. The function of the lock and release is therefore identical with the first embodiment discussed above previously.

In one embodiment, the cannula cover 7 is provided with a two-step catch 25 having a narrower, wedge shaped step 26 and a wider, block-shaped step 27 and in other embodiments, with two such catches 25 that may be arranged opposed each other along the circumference of the cover 7. In still other embodiments, two or more of such catches 25 may be provided around the circumference of the holder 4. In some of the embodiments, each catch 25 is formed at the end of an elastic tongue 28, which allows the catch to be bent in inward direction of the cannula cover, as seen in FIG. 11 and to spring back into its rest position shown in FIGS. 6, 8 and 10. This tongue 28 and the catch 25 can be in one piece with the wall 29 of the cannula cover 7. Further protruding ribs 30 may be provided on the outside of this wall 29 to provide sliding guides for the cannula cover 7 within the holder 4 that is provided in this case with corresponding groves or slits taking up these sliding guides. The cannula holder 4 is provided with the hole 31 for taking up the block-shaped step 27 of catch 25 (and in the case of several catches/tongues is provided with the same number of corresponding holes 31 for the block-shaped steps of these catches).

When the cannula cover 7 is mounted in the cannula holder 4 and the cover 7 is in the locked state covering the cannula, then catch block-shaped step 27 of catch 25 fits in the correspondingly shaped hole 31 and the cannula cover is thus locked against movement in this position, for example, as shown in FIGS. 1 and 6. When the cannula holder 4 is inserted into the receiving element 10, then the release will remove the catch from this locked position, allowing the moving of the cannula cover 7 during the insertion movement of the cannula holder 4 into the receiving element 10. In the shown example, the rib 16 (and in the case of several catches 25, the correspondingly placed several ribs 16) on the inside of the receiving element 10 first slides within a groove 32 of the cannula holder 4 leading to the hole 31. Then, when the insertion of the cannula cover 7 into the receiving element 10 proceeds, rib 16 contacts the wedge shaped, narrower step 26 of catch 25 that protrudes into groove 32 (as explained before with reference to FIG. 10). The rib 16 then forces the catch 25 inwardly (in direction towards the cannula 5), thereby elastically deforming tongue 28. By this inwardly forcing, block-shaped step 27 frees from hole 31 and the cover 7 can slide upwards (away from the cannula tip). During the upward movement, step 26 of catch 25 is sliding within slit 33 of the cannula holder 4 that connects to hole 31.

When the cannula cover 7 is fully moved backwards (which, as well, corresponds with the blocking of the cannula cover in the receiving element 10 by the blocking element 20 explained above), the position of FIG. 11 is taken, wherein the inwardly bent tongue 28 and step 26 and rib 16 can be seen. When the blocking elements 20 are unblocked and the cannula holder is pulled out of receiving element 10, rib 16 moves accordingly downwards (in direction towards the tip of the cannula 5) and the cannula cover 7 is pressed downwardly by coil spring or energy storage device 8 as well. During this downward movement, catch 25 again reaches hole 31 where the block-shaped step 27 of catch 25 again enters the hole due to the spring back elasticity of the tongue 28. Thus, cover 7 has moved, during the pull out movement, over the cannula 5, thereby covering same more and more until the cover 7 is again locked in the state of fully covering the cannula when the cannula holder 4 is fully removed from the receiving element 10, and thus from device 2. Thus, a fool-prove cannula cover results with a fully automatic function both during insertion of the cannula holder into and pulling out of the cannula holder from the receiving element, and thus from device 2. In the fully inserted state shown in FIG. 12, the cannula 5 has pierced the reservoir 3 (and in case of an ampoule, the septum 39 thereof) so that a fluid connection between reservoir 3 and line 6 is provided by means of the cannula 5.

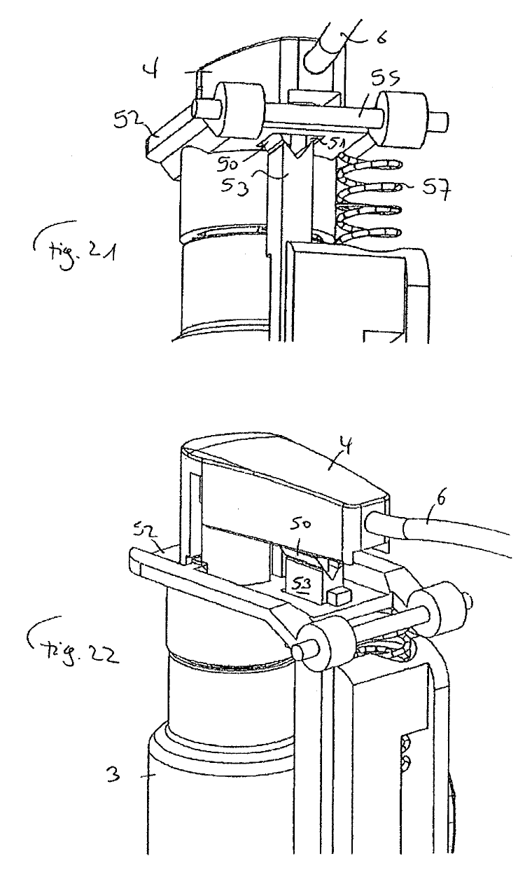

The blocking and unblocking elements of this embodiment could have been selected identical to the blocking and unblocking elements of the first embodiment but have been selected differently from the first embodiment. Here, two latches 50 and 51 are provided on the lower outside of the horizontal part of the L-shaped cannula holder 4, as can be seen in FIGS. 19 and 20. When the cannula holder 4 is inserted into the receiving element 10, these latches 50 and 51 snap into a hole 56 within plate 52 of the cover when the cannula holder 4 is fully inserted into device 2, as can be seen in FIG. 21. To unblock the cannula holder 4, the two latches 50 and 51 are pressed together by a V-shaped lifter 53 that can be manually actuated by a knob on the outside of the pump housing against the force of spring 57. In this manner, the latches 50 and 51 come free of plate 52, which are then pressed upwardly to unblock the cannula holder 4. FIG. 22 shows the action of the lifter 53 that has already pushed the latches 50 and 51 away from the plate 52.

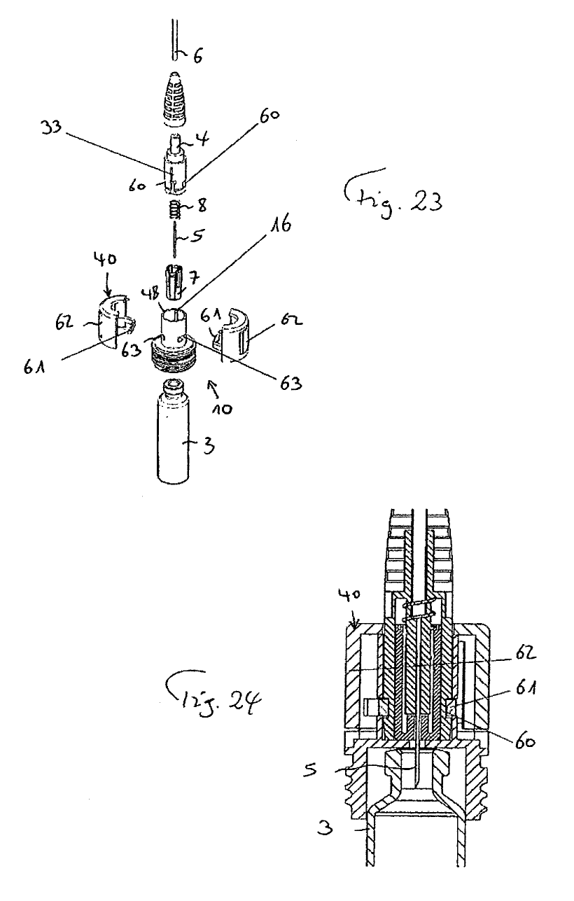

With reference to FIGS. 23 and 24, a further embodiment is described with different means for blocking and unblocking of the cannula holder. FIG. 23 shows an exploded view of the cannula holder 4 and the receiving element 10 which is here again of the adapter type. The cannula holder, the lock, and the release for the cannula cover are of the same construction as described for the first embodiment and reference is made to this description above. The blocking and unblocking elements, on the other hand, may comprise at least one recess 60 in the outer wall of cannula holder 4 and at least one corresponding finger 61 on the adapter or receiving element 10 that is arranged to reach into the recess 60 when the cannula holder 4 is fully inserted into the adapter or receiving element 10. In this manner, the cannula holder is blocked within the adapter 10 by the finger. The embodiment shown in FIGS. 23 and 24 comprises two recesses 60 and two corresponding fingers 61. The fingers 61 reach so far through slit-shaped holes 63 in the wall of seat 48 of adapter or receiving element 10 so that they can enter the recesses 60. On the other hand, the fingers 61 are connected to knobs 62 that can be pressed together by the user. By this pressure, the fingers 61 make a movement away from the cannula holder 4 and its recesses 60 and the cannula holder 4 is unblocked and will be pushed out of seat 48 and thus out of adapter or receiving element 10 by the force of the spring or energy storage device 8.

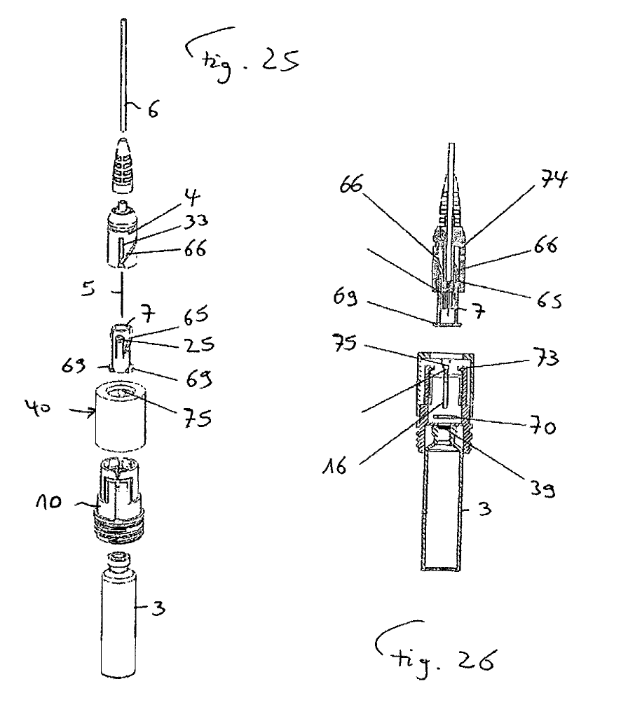

FIGS. 25 and 26 show another embodiment wherein no energy storage element 8 is provided. In this embodiment, the cannula cover 7 is provided with a screw thread shaped protrusion 65 that is guided in a corresponding screw thread shaped guiding slit 66 in the wall of the cannula holder 4. Thus the cannula cover 7 will be move backwards in a rotating movement when the lock here provided by at least one catch 67 (similar to the catch 25 of the other embodiments) are unlocked by the release which are provided here as well by a rib or ribs 16. The rib or ribs 16 prevent, on the other hand, a turning of the cannula holder 4 with the guiding slit. In this manner, the cannula cover 7 when pushed backwards only rotates by abutting either on the adapter ground 68 or on the ampoule 3 while the cannula holder 4 is inserted into the adapter or receiving element 10 and the screw thread-shaped guide 65, 66 forces the rotating movement. The cannula 5 is exposed by the rotating backwards movement of the cannula cover 7. As well, by the rotating movement of the cannula cover 7, noses 69 on this cover rotate and enter by this turning movement below protrusions 71 on the inside wall of seat 48 of the adapter or receiving element 10. The cannula cover 7 is therefore held within adapter or receiving element 10 against pulling out from the adapter or receiving element and thus, when the cannula holder 4 is again pulled out from the adapter or receiving element 10, the cannula cover 7 is pulled over the cannula 5 again by making the rotating movement by the guide 65, 66 in opposite direction compared to the insertion. By this rotating movement, the cannula cover 7 is finally free again from protrusions 71 since noses 70 turn free from these protrusions by the rotating movement as well, so that the cannula holder 4 with the cover 7 can be pulled out of the adapter or receiving element 10. The blocking and unblocking elements of this embodiment can be similar to the corresponding means of the first embodiment. Different blocking and unblocking elements are shown in FIGS. 25 and 26. Catches 73 are provided on the adapter or receiving element 10 that are arranged to fit into recesses 74 on the outside of the cannula holder 4 when this holder is fully inserted into the adapter or receiving element 10. A wedge 75 on the unblocking element is adapted to force the catches 73, which are correspondingly wedge-shaped, out of the recesses 74 when the cannula holder 4 shall be unblocked from the receiving element 10, as has generally be explained before. The details of such a blocking element and an unblocking element are not shown for this embodiment, since the detail construction of such means can easily be conceived by the skilled person.

What is claimed is:

1. A connection system for connecting a fluid line to a fluid reservoir located within a device by way of a cannula, comprising:
a cannula holder holding the cannula and comprising a movable cannula cover and a lock for said cover which prevents the movement of the cannula cover in a first state covering the cannula;
a receiving element which receives the cannula holder, the receiving element being adapted for fixation to the device or the receiving element forming part of the device, said receiving element comprising a release which interacts with the lock, and wherein
the release and the lock are arranged to release the cannula cover by said release interacting with the lock, so that the cannula cover is movable by the inserting force to expose the cannula when the cannula holder is inserted into the receiving element, so that the cannula cover is in a second state exposing the cannula when the cannula holder is inserted within the receiving element.

2. A connection system according to claim 1 wherein as the cannula holder is removed from the receiving element the cannula cover is moved by an energy storage device in a direction opposite to the movement during insertion.

3. A connection system according to claim 1 wherein the cannula cover is held slidably within the cannula holder for a sliding translation movement in longitudinal direction of the cannula.

4. A connection system according to claim 1 wherein the cannula cover is held within the cannula holder by a guide imparting a rotational screw-in and screw-out movement to said cannula cover within the cannula holder during the insertion and the removing of the cannula holder from the receiving element.

5. A connection system according to claim 1 wherein the lock comprise a catch on the cannula cover which locks into a recess of the cannula holder and the release comprises a rib within the receiving element which releases the catch from the recess.

6. A connection system according to claim 5 wherein the catch is a two-stepped catch comprising a first block-shaped step and a second step which is held by an elastically deformable tongue.

7. A connection system according to claim 1 comprising a blocking element and an unblocking element for releasably blocking the cannula holder within the receiving element and in particular comprising unblock provided for one-handed operation of the means.

8. A connection system according to claim 1 wherein the receiving element is part of a medical device and in particular part of an insulin pump.

9. A connection system according to claim 1 wherein the cannula holder is essentially L-shaped and wherein the cannula is held essentially in a 90 degree angle to the line exiting the cannula holder.

10. A connection system according to claim 8 wherein the receiving element is mounted to the device such that it swivels about an axis for opening a chamber adapted to receive the liquid reservoir.

11. A medical pumping device with a receiving element according to claim 1.

12. A medical pumping device according to claim 11 which is a portable medication pump.

13. A medical pumping device according to claim 11 which is an insulin pump.

14. A connection system according to claim 2 wherein the energy storage device is a coil spring.

15. A method of connecting a fluid line to a fluid reservoir located within a device by way of a cannula, comprising utilizing the connecting system of claim 1.
